(19) United States
(12) Patent Application Publication
(10) Pub. No.: US 2021/0077710 A1
(43) Pub. Date: Mar. 18, 2021

(54) AUTO INJECTOR SET FOR OXYGEN REDUCED PACKAGING

(71) Applicant: Inject Pharma Sweden AB, Bromma (SE)

(72) Inventor: Sebastian Helmut Jose Bauer, Enskede (SE)

(21) Appl. No.: 16/956,225

(22) PCT Filed: Dec. 21, 2018
(86) PCT No.: PCT/EP2018/086673

§ 371 Date: Jun. 19, 2020

(30) Foreign Application Priority Data
Dec. 21, 2017 (SE) 1751618-8

(51) Int. Cl. A61M 5/00; A61M 5/20; B65B 31/04; B65D 85/20

(52) U.S. Cl. CPC A61M 5/002; A61M 5/20; A61M 2005/3123; B65D 85/20; B65B 31/04

(57) ABSTRACT

The present invention relates to a packaged auto injector set. It comprises a partly transparent medicament container, a hollow auto injector body with the medicament container forming the auto injector, and a non-oxygen permeable package (2) with an opening arrangement (3) for opening the package (2). The auto injector body has at least two apertures (4) allowing for visual control of the medical composition, at least a part of the package (2) is transparent visual control of the medical composition and the packaged auto injector set comprises at least one blocking arrangement (5), which is non-transparent to at least ultraviolet light. The present invention also relates to a method for filling a package (2) with an auto injector. The method comprises inserting the auto injector into the package (2) in a non-inert environment and under atmospheric pressure, replacing the air in the package (2) with inert atmosphere by one or several cycles of removing air and inserting inert gas through the opening. The opening is sealed under vacuum.

### TECHNICAL FIELD

The present invention relates to an auto injector for administrating adrenaline and a method for filling a package with the auto injector.

### BACKGROUND

Auto injectors are intended to be used to self-administer drugs. There are many different kinds of designs of auto injectors but the common denominator is that they are for overcoming the hesitation associated with self-administrating drugs. They are usually a needle-based device with a pre-loaded syringe. The needle is shielded before use so that it is not possible to hurt oneself on it.

When designing an auto injector, it is not only the functionality that needs to be considered. The usability is as important since an auto injector is often used by people untrained in using them. It needs to be very clear how to use it to a person picking up the device. For example, epinephrine auto injectors, for use by people who are at risk for anaphylaxis, are often used under time pressure when it is important that the patient receives the injection quickly after an allergen exposure. In such cases it is even more crucial that the auto injector is easy to use, and self-explanatory in its design.

Another issue with designing auto injectors is that the life span of the drug is affected by the design. For example, some drugs are very sensitive to oxygen, all drugs are sensitive to plunger movement at transportation, because sterility can be compromised, and some are sensitive to light. Oxidative degradation is a chemical process that renders many drugs inactive, by degrading the active ingredient, or makes the product unusable, by changing the properties of the excipients or by changing the physical properties, parts or all. Furthermore, oxidation can also have a negative impact on plastic components of the auto injector and thus reduce reliability and shelf life of the mechanical auto injector.

Auto injectors are often packaged in a protective packaging to protect them before use. For drugs that are sensitive to oxygen and/or other atmospheric gases, the process of packaging the auto injector is very important and often onerous.

There is a need for simplifying the process of packaging auto injectors and at the same time providing an auto injector that is easy to use and that displays a long shelf life for the medicament.

### SUMMARY

It is an aim of the present invention to at least partly overcome the above problems, and to provide an improved auto injector and a method for packaging of auto injectors.

The present disclosure aims to provide a packaged auto injector with increased usability and a more user-friendly packaging process of the auto injector.

This aim is achieved by the device as defined in claim 1 and the method defined in claim 14.

According to an embodiment of the disclosure, it comprises a packaged auto injector set for providing a packaged auto injector with an adrenaline composition. The auto injector set comprises an at least partly transparent medicament container comprising an adrenaline composition, a hollow auto injector body defining a space which houses the medicament container and thereby forming the auto injector, and a non-oxygen permeable package for housing the auto injector and including an opening arrangement for opening the package to retrieve the auto injector. The auto injector body is provided with at least two apertures disposed on opposite sides of the medicament container allowing for visual control of the adrenaline composition inside the container, at least a part of the package is transparent to visible light to allow visual control of the adrenaline composition through the apertures, and the packaged auto injector set comprises at least one blocking arrangement, which is non-transparent to at least ultraviolet light, arranged such that ultraviolet light is prevented from entering said at least two apertures. Thus, the packaged auto injector set allows for visual control of the adrenaline composition and the blocking arrangement protects the drug from ultraviolet light. It should be noted that the packaged auto injector set can be used with any medical composition and not only adrenaline. Exposure to ultraviolet light may lead to decomposing or molecular changes of the medical composition. In other words, ultraviolet light is prevented from entering the two apertures so that it cannot affect the adrenaline.

According to some aspects, the at least one blocking arrangement is arranged on the auto injector body and comprises at least one film placed over the at least two apertures, wherein the film is transparent to visible light and non-transparent to ultraviolet light. This arrangement makes sure that ultraviolet light is kept from entering the two apertures both when the auto injector is packaged and unpackaged. In other words, it protects the medical composition before packaging, when it is packaged and after opening the package and retrieving the auto injector.

According to some aspects, the at least one blocking arrangement is arranged on the part of the package which is transparent to visible light and comprises a layer in the transparent part which is transparent to visible light and non-transparent to ultraviolet light. Thus, ultraviolet light is prevented to enter the two apertures, but the package is still see through to allow for visual control of the medical composition. This kind of blocking arrangement blocks ultraviolet light without the need of a blocking arrangement on the auto injector.

According to some aspects, the at least one blocking arrangement is arranged on the package and wherein the transparent part of the package is closable such that the package is non-transparent to visible light and ultraviolet light when the transparent part is closed and thus blocks visible light and ultraviolet light from entering the at least two apertures. Depending on the medical composition, there may be differences in how sensitive it is to ultraviolet light. Some medical compositions are also sensitive to visible light, such as adrenaline. So, the blocking arrangement may be arranged to prevent both ultraviolet and visible light from entering the apertures.

According to some aspects, the transparent part is arranged on a first side of the package and the package has a second side, which is non-transparent to ultraviolet light and visible light, wherein the blocking arrangement comprises a folding of the second side of the package over the first side of the package to cover the transparent part and thus blocking ultraviolet light and visible light from entering the at least two apertures. In other words, the package is foldable and when it is folded, a non-transparent side is covering the transparent side. It should be noted that if the transparent part is large, the fold may not cover the whole transparent area, but the non-transparent part must at least cover the transparent part which is located over the two apertures.

According to some aspects, the opening arrangement is located such that it is not covered when the package is folded. That is, the opening arrangement should not be blocked by the blocking arrangement.

According to some aspects, the opening arrangement is arranged to be torn by a user such that the package tears, and wherein the opening arrangement comprises a stop that prevents the user from tearing the package all the way through, such that the package is kept in one part when teared. A stop is for example a thick part of the package which is not easily torn through. The stop may also be another material in the package that prevents tearing. There is a significant risk that the auto injector is dropped by the user if the opening of the package is not controlled. It the user tears the package and it easily opens all the way through, the user may not be prepared to catch the auto injector. The opening arrangement is for example a pre-notch or two sides of the package adhered together such that they are to be torn apart from each other.

According to some aspects, the stop comprises an increased tear resistance in the package. Tear resistance may be affected in several ways; for example, with a thicker part of the package or with another material making it harder to tear through the package.

According to some aspects, the opening arrangement comprises a primary opening arrangement for opening the package and a secondary opening arrangement for opening the package in case of failure to open with the primary opening arrangement. Since safe and fast retrieving of the auto injector may be crucial to a patient, a secondary opening arrangement may be used as a back-up should there be a problem with the primary opening arrangement. The primary and secondary opening arrangements may be different types of opening arrangements or the same.

According to some aspects, the opening arrangement comprises a pre-notch. Pre-notches are easily understood by users so that it is self-explanatory for a user how to open the package to retrieve the auto injector.

According to some aspects, the package is in the shape of a rectangle and the pre-notch is located on one of the longer sides of the rectangle. If the notch is located on a short side, the opening of the package may result in an opening that is too small to retrieve the auto injector from.

According to some aspects, the package comprises two sides, a first and a second side, which are attached to each other and wherein the two sides are openably attached such that the user tears the two sides apart when opening the package. Such an opening arrangement is also something that users have seen before and can easily use. The two sides are for example welded together.

According to some aspects, the package comprises an upper half and a lower half and wherein the auto injector body comprises a first end comprising a cap and a second end, the auto injector being housed in the package such that the first end is located in the lower half of the package and the opening arrangement being arranged on the upper half of the package. This is such that the cap side of the auto injector is not the end that is pulled out first of the package by the user, to prevent that the cap is accidentally opened when extracting the auto injector.

According to some aspects, the un-opened package is filled with inert gas and is free of oxygen capturing substance. Not having to use an oxygen capturing substance to capture residual oxygen in the packaging is a consequence of the packaging method herein disclosed. This saves cost and material.

According to some aspects, the adrenaline composition comprises a chemical oxygen scavenger. Chemical oxygen scavengers are used to prevent oxidative degradation of the medical composition. It is used to further increase the shelf life of the auto injector.

According to some aspects, the chemical oxygen scavenger comprises sodium metabisulfite. Sodium metabisulfite is an effective chemical oxygen scavenger.

According to some aspects, the concentration of sodium metabisulfite in the adrenaline composition is below or equal to 0.21 mg/ml. It is desirable to use as little sodium metabisulfite as possible since it can affect the medical composition.

According to some aspects, the auto injector body is elongated having an elongated part, a first end side and a second end side opposite the first end side, and at least one through hole is arranged through the auto injector body between the first and second end sides, whereby the at least one through hole allow for flow of gas through the auto injector body when the auto injector body houses the medicament container, and the package is a pouch and the inside of the pouch is at least 70 mm longer than the total length of the auto injector and has a maximum width, at the most narrow part of the pouch, of 30 mm wider than the maximum width of the auto injector. An advantage with an auto injector which has through holes between the end sides is that the package can be made much more narrow than conventional packages. This is because air behind the auto injector will be evacuated through the through holes and not via the sides of the auto injector. This reduces the amount of packaging needed and it also makes the packaged auto injector easier to handle for the end user.

According to some aspects, the package filled with the auto injector is snugly arranged in a neoprene sleeve such that the movement of the auto injector relative the package is prevented. The neoprene sleeve protects the packaged auto injector from physical damage, keeps the temperature of the auto injector more constant and minimizes movement of the packaged auto injector and thus minimizes friction which can lead to punctures. In the case where the auto injector with through holes and the narrower pouch is used for package, there will not be a lot of packaging material around the auto injector. It is easy to store this packaged auto injector set in a neoprene sleeve; there is not a lot of package that needs to be held in when putting it in the sleeve.

The disclosure further comprises a method for filling a package with an auto injector comprising an adrenaline composition, the package comprising an opening. The adrenaline composition may comprise a chemical oxygen scavenger. The method comprises inserting the auto injector into the package via the opening in a non-inert environment and, in a non-inert environment and under atmospheric pressure, replacing the air in the package with inert atmosphere by one or several cycles of removing air and inserting inert gas through the opening. The method also comprises sealing the opening under vacuum. This method has a big advantage that it can be performed in a non-inert environment. To keep an inert environment for packaging is very costly and cumbersome.

According to some aspects, the air is removed using a nozzle type vacuum sealing machine. Such a machine has a nozzle that is inserted in the opening of the package and removes the air through the nozzle. This type of machine provides a local point in the opening for removing the air which is an effective way of only removing the air that is in the package.

### BRIEF DESCRIPTION OF THE DRAWINGS

The invention will now be explained more closely by the description of different embodiments of the invention and with reference to the appended figures.

FIG. 1 shows an example of an auto injector showing the apertures

FIG. 2 shows an example of a packaged auto injector set showing that the apertures are visible through the package

FIG. 3 shows an example of a package with a pre-notch opening

FIG. 4 shows an example of a package with a tear opening

FIG. 5 shows an example of a package with a primary and secondary opening

FIG. 6 shows an example of a package with a primary, secondary and tertiary opening

FIG. 7 shows an example of a packaged auto injector set where the arrow indicates how to fold the package to close the blocking arrangement

FIG. 8 shows an example of a packaged auto injector set where the package has been folded

FIG. 9 shows an example of a packaged auto injector set where the package has been folded in two places

FIG. 10 shows an example of a cut through of an auto injector with enlarged parts

FIG. 11 shows an example package for the auto injector

FIG. 12 shows a block diagram of the method

FIG. 13 shows an example shape of a package for the auto injector

FIG. 14 shows an example shape of a package for the auto injector

FIG. 15 shows an example of a nozzle type vacuum sealing machine

### DETAILED DESCRIPTION

Aspects of the present disclosure will be described more fully hereinafter with reference to the accompanying drawings. The device and method disclosed herein can, however, be realized in many different forms and should not be construed as being limited to the aspects set forth herein. Like numbers in the drawings refer to like elements throughout.

The terminology used herein is for the purpose of describing particular aspects of the disclosure only and is not intended to limit the invention. As used herein, the singular forms “a”, “an” and “the” are intended to include the plural forms as well, unless the context clearly indicates otherwise.

Unless otherwise defined, all terms used herein have the same meaning as commonly understood by one of ordinary skill in the art to which this disclosure belongs.

The term “adrenaline composition” is defined as compositions comprising adrenaline, also known as epinephrine, and salts thereof. Such salts include, but are not limited to, adrenaline tartrate and adrenaline hydrochloride.

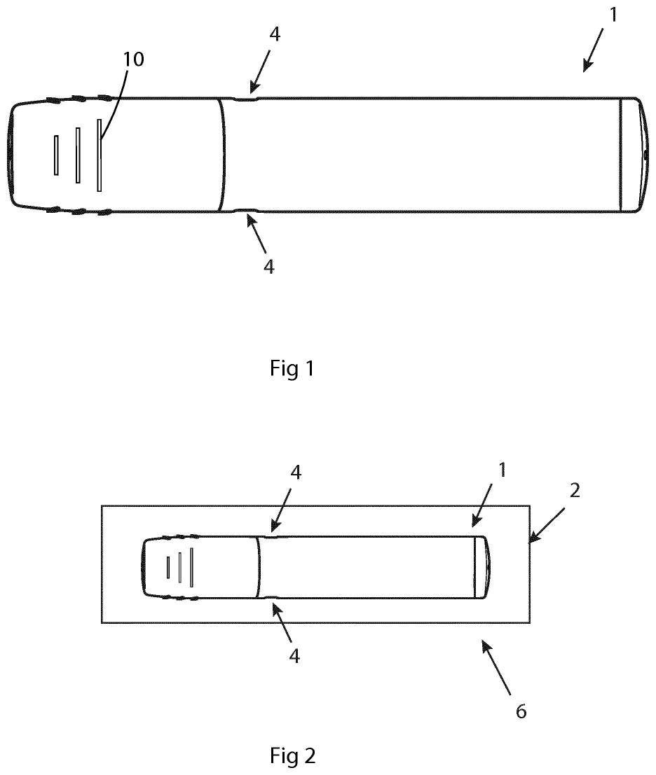

FIG. 1 shows an illustration of an auto injector 1 showing two apertures 4 for visual control of the drug inside and FIG. 2 illustrates when such an auto injector is packaged in a package 2. There are also grips 10 on the sides of the auto injector for giving a good grip to the user when handling the auto injector. The grips are for example rubber ridges.

The disclosure provides a packaged auto injector set 6 for providing a packaged auto injector 1 with an adrenaline composition. The auto injector set comprises an at least partly transparent medicament container comprising an adrenaline composition. Other common terms for the medicament container are primary packaging and primary containers. The medicament container is for example a syringe. The syringe may be spring loaded to push it out when injecting the drug. The medicament container is for example a prefilled syringe with a staked-on needle and having permeability to oxygen, e.g. a prefillable syringe with a staked-on needle and a rigid or flexible needle shield, which is sterilized by EtO and thus permeable for oxygen. Oxygen permeable primary containers are very common, since sterilization is often performed by EtO gas which requires gas permeability of e.g. the needle shield. Other sterilization methods have other disadvantages. Radiation sterilization discolors glass, autoclavation does only allow for use of bulk syringes. There are many types of medicament containers that can be used in an auto injector and for this disclosure, it is not relevant which one is used. It should be noted that other medical compositions than adrenaline may be used in the disclosed packaged auto injector set 6. The packaged auto injector set according to this invention is suitable for use with adrenaline because it is easy to open, easy to use and gives a long shelf life to the auto injector. In other words, the auto injector is an epinephrine auto injector.

The primary container may be filled, aseptic or sterilized by terminal sterilization, with drug product either bubble free, if drug viscosity allows, or containing an inert gas bubble. The drug formulation can contain an oxygen scavenger, such as sodium metabisulfite, ascorbic acid etc., if residual oxygen, which is generally unavoidable during filling and packaging, has to be scavenged. Thus, according to some aspects, the adrenaline composition comprises a chemical oxygen scavenger. Chemical oxygen scavengers are used to prevent oxidative degradation of the medical composition. Oxidative degradation is a chemical process that renders many drugs inactive by degrading the active ingredient or makes the product unusable by changing the properties of the excipients or by changing the physical properties of the liquid container or of the device. It is used to increase the shelf life of the auto injector 1. According to some aspects, the chemical oxygen scavenger comprises sodium metabisulfite. Sodium metabisulfite is an effective chemical oxygen scavenger. According to some aspects, the concentration of sodium metabisulfite in the adrenaline composition is below or equal to 0.21 mg/ml, such as below 0.2, 0.18or 0.16 mg/ml). Preferably, the concentration of sodium metabisulfite in the adrenaline composition is below or equal to 0.15 mg/ml, such as below 0.14 or 0.12 mg/ml. More preferably, the concentration of sodium metabisulfite in the adrenaline composition is below or equal to 0.1 mg/ml, such as below 0.08 or 0.06 mg/ml. Even more preferably, the concentration of sodium metabisulfite in the adrenaline composition is below or equal to 0.05 mg/ml, such as below 0.04 or 0.02 mg/ml. It is desirable to use as little sodium metabisulfite as possible since it can affect the medical composition, provided that an acceptable and/or desired shelf life can be achieved. A reason is that the sodium metabisulfite breaks down adrenaline. Sulfites can cause allergy like reactions (intolerances); most commonly asthma symptoms in those with underlying asthma, sometimes allergic rhinitis (hay fever) like reactions, occasionally urticaria (hives) and very rarely, anaphylaxis (severe allergic reactions) or even anaphylactic shock. Wheezing is the most common reaction. Oxygen scavenger concentration can be reduced/minimized by preventing oxygen ingress. This can be advantageous if the scavenger also contributes to chemical degradation of the active ingredient, which is true for e.g. adrenaline in combination with e.g. sodium metabisulfite, or if authorities demand a reduction of preservative amounts. The auto injector 1 and method for packaging disclosed in this document minimizes, and may even remove the need for, the use of oxygen scavengers as will be further explained below.

A hollow auto injector body defines a space which houses the medicament container and thereby forming the auto injector 1. The shape of the auto injector body can be adapted to house different kinds of medicament containers. The auto injector body is, according to some aspects, also adapted to house other parts of the auto injector 1, such as a spring, if there is a spring-loaded syringe. Another example is that the auto injector body may house a pressurized gas container if the auto injector is a gas jet auto injector which uses pressurized gas to propel a fine jet of the drug through the skin of the patient without using a needle.

It should be noted that other types of medical injection devices than auto injectors may be used within the scope of the disclosure. In other words, the content of the disclosure can be applied to any medical injection device, including auto injectors.

The auto injector is packaged in a non-oxygen permeable package 2 for housing the auto injector and including an opening arrangement 3 for opening the package 2 to retrieve the auto injector. Wit a non-oxygen permeable package means a package that allows for less than 1 ml O₂ over 3 years. Preferably, a non-oxygen permeable package means a package that allows for less than 0.1 ml O₂ over 3 years. It is nearly impossible to provide a completely non-oxygen permeable package.

The auto injector body is provided with at least two apertures 4 disposed on opposite sides of the medicament container allowing for visual control of the adrenaline composition inside the container. Two, or more, control windows, i.e. apertures, will allow for visual control with light from behind to improve the controllability of e.g. particles and/or discoloration. Visual control of the medical composition, i.e. the drug inside the auto injector, is important so that the user can easily check if there is something wrong with the drug; for example, if the container is broken such that the drug is leaking out or that the drug has changed colour, for example due to oxygen exposure or particle formation.

At least a part of the package 2 is transparent to visible light to allow visual control of the adrenaline composition through the apertures 4, and the packaged 2 auto injector set 6 comprises at least one blocking arrangement 5, which is non-transparent to at least ultraviolet light, arranged such that ultraviolet light is prevented from entering said at least two apertures 4. The blocking arrangement 5 can be arranged either on the auto injector body or the package. Thus, the packaged auto injector set allows for visual control of the medical composition and the blocking arrangement 5 protects the drug from ultraviolet light. Exposure to ultraviolet light may lead to photolytic degradation changes of the medical composition. In other words, ultraviolet light is prevented from entering the two apertures so that it cannot affect the medical composition.

The blocking arrangement 5 can be achieved in some different ways. According to some aspects, the at least one blocking arrangement 5 is arranged on the auto injector body and comprises at least one film placed over the at least two apertures 4, wherein the film is transparent to visible light and non-transparent to ultraviolet light. The film may for example be a sticker that is adhered to the auto injector body over the apertures. The film is either one film large enough to cover both apertures or two films, one for each aperture. Alternatively, the film is part of an auto injector label so that UV protection and labelling happens in one step. This arrangement makes sure that ultraviolet light is kept from entering the two apertures both when the auto injector is packaged and unpackaged. In other words, it protects the medical composition before packaging, when it is packaged and after opening the package 2 and retrieving the auto injector.

Another example of a blocking arrangement 5 is that the at least one blocking arrangement 5 is arranged on the part of the package 2 which is transparent to visible light and comprises a layer in the transparent part which is transparent to visible light and non-transparent to ultraviolet light. Thus, ultraviolet light is prevented to enter the two apertures 4 but the package 2 is still see through to allow for visual control of the medical composition. This kind of blocking arrangement 5 blocks ultraviolet light without the need of a blocking arrangement 5 on the auto injector.

Depending on the medical composition, there may be differences in how sensitive it is to ultraviolet light. Some medical compositions are also sensitive to visible light. So, the blocking arrangement 5 may be arranged to prevent both ultraviolet and visible light from entering the apertures 4. According to some aspects, the at least one blocking arrangement 5 is arranged on the package 2 and wherein the transparent part of the package 2 is closable such that the package 2 is non-transparent to visible light and ultraviolet light when the transparent part is closed and thus blocks visible light and ultraviolet light from entering the at least two apertures 4. An example aspect would be a pouch consisting of two different foils, welded together to form a pouch. Such a pouch is depicted in FIGS. 7-9 in a way that the non-transparent foil is below the autoinjector, hence the visibility of the auto injector (unless parts have been folded in that case the non-transparent parts are depicted black and covered parts of the auto injector have become invisible in the figures).

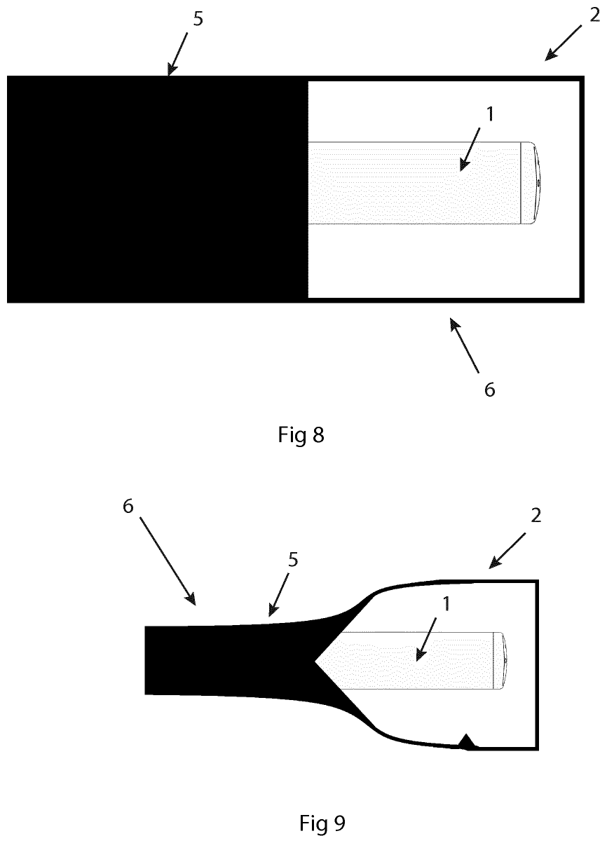

There are different ways to provide a closable transparent part. According to some aspects, the transparent part is arranged on a first side of the package 2 and the package 2 has a second side, which is non-transparent to ultraviolet light and visible light, wherein the blocking arrangement 5 comprises a folding of the second side of the package 2 over the first side of the package 2 to cover the transparent part and thus blocking ultraviolet light and visible light from entering the at least two apertures 4. In other words, the package is foldable and when it is folded, a non-transparent side is covering the transparent side. Such an example is illustrated in FIGS. 7 and 8, where FIG. 7 illustrates the package before folding and FIG. 8 after folding. In the illustrated examples, one side of the package is transparent and the other is non-transparent. It should be noted that if the transparent part is large, the fold may not cover the whole transparent area but the non-transparent part must at least cover the transparent part which is located over the two apertures 4. This is the case in the example of FIGS. 7 and 8. Another example of a closable transparent part is that the transparent part comprises two transparent parts on two sides of the package arranged such that each transparent part is located over an aperture of the auto injector body, and the blocking arrangement 5 comprises a folding of the package in two places such that the transparent parts are covered with parts of the package that are non-transparent to ultraviolet light and visible light and thus blocking ultraviolet light and visible light from entering the at least two apertures. Another example is illustrated in FIG. 9, where two sides of the package are folded in over the auto injector as shown in the figure.

Folding of the package 2 should not affect the accessibility to the auto injector, so, according to some aspects, the opening arrangement 3 is located such that it is not covered when the package 2 is folded. That is, the opening arrangement 3 should not be blocked by the blocking arrangement 5. This may be realized by designing the package 2 such that it is not folded in the middle but is biased to a side of the package. Thus, not the whole package is in the folded part. In FIG. 9, an opening arrangement in the form of a pre-notch is illustrated which is at the opposite end of the package than the blocking arrangement.

The blocking arrangement 5 may also be arranged as a flap covering the transparent part. Thus, according to some aspects, the at least one blocking arrangement 5 comprises a flap arranged over the transparent part, whereby the flap is adapted to be lifted to allow visual control of the adrenaline composition. According to some aspects, the transparent part comprises two transparent parts on two sides of the package 2 and wherein the at least one blocking arrangement 5 comprises two flaps arranged respectively over the transparent parts, whereby each flap is adapted to be lifted to allow visual control of the medical composition.

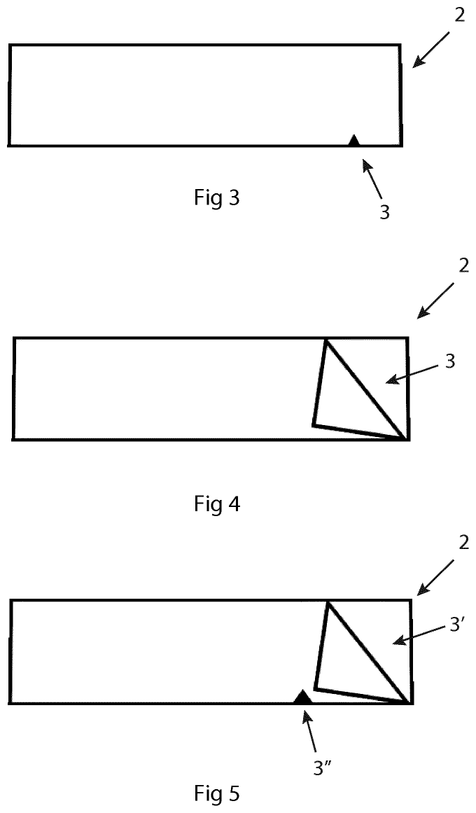

There is a significant risk that the auto injector is dropped by the user if the opening of the package 2 is not controlled. If the user tears the package and it easily opens all the way through, the user may not be prepared to catch the auto injector. It is common to use medicament containers made of glass and they can be especially sensitive to dropping the auto injector. Plastic medicament containers may be more durable but may pose other problems with the purity of the drug located inside it. According to some aspects, the opening arrangement 3 is arranged to be torn by a user such that the package 2 tears, and wherein the opening arrangement 3 comprises a stop that prevents the user from tearing the package 2 all the way through, such that the package 2 is kept in one part when teared. A stop is for example a thick part of the package 2 which is not easily torn through. The stop may also be another material in the package that prevents tearing. The opening arrangement 3 is for example a pre-notch or two sides of the package adhered together such that they are to be torn apart from each other. When the opening arrangement 3 is two sides tearably adhered together the stop may be that the adhesive is different and stronger at the stop than at the tearable part. According to some aspects, the package 2 comprises two sides, a first and a second side, which are attached to each other and wherein the two sides are openably attached such that the user tears the two sides apart when opening the package 2. Such an opening arrangement 3 is also something that users have seen before and can easily use. The peel strength of such a solution may be for example around 0.18 kg/cm or 1 lb./inch. FIG. 3 illustrates an example of a package 2 with a pre-notch opening and FIG. 4 shows an example package 2 with a tear opening.

The stop comprises, according to some aspects, an increased tear resistance in the package 2. The tear resistance shall be optimized to minimize risk that pouch is torn apart fully to minimize the risk that the auto injector falls to the ground. Tear resistance may be affected in several ways; for example, with a thicker part of the package 2, with another material making it harder to tear through the package 2 or with a strong adhesive as mentioned above. An alternative to the stop is that it comprises a sticker put on the package 2 such that it hinders the package 2 from opening at the sticker.

As previously discussed, the opening arrangement 3 comprises, for example, a pre-notch. Pre-notches are easily understood by users so that it is self-explanatory for a user how to open the package 2 to retrieve the auto injector. According to some aspects, the package 2 is in the shape of a rectangle and the pre-notch is located on one of the longer sides of the rectangle. If the notch is located on a short side, the opening of the package 2 may result in an opening that is too small to retrieve the auto injector from. A package that has been adjusted to a specific auto injector may have a minimized size which leads to a short end which is ca ½-⅓ of the long side of the package. The shape of the package may be a rectangle and it may have rounded corners giving an oval-like shape.

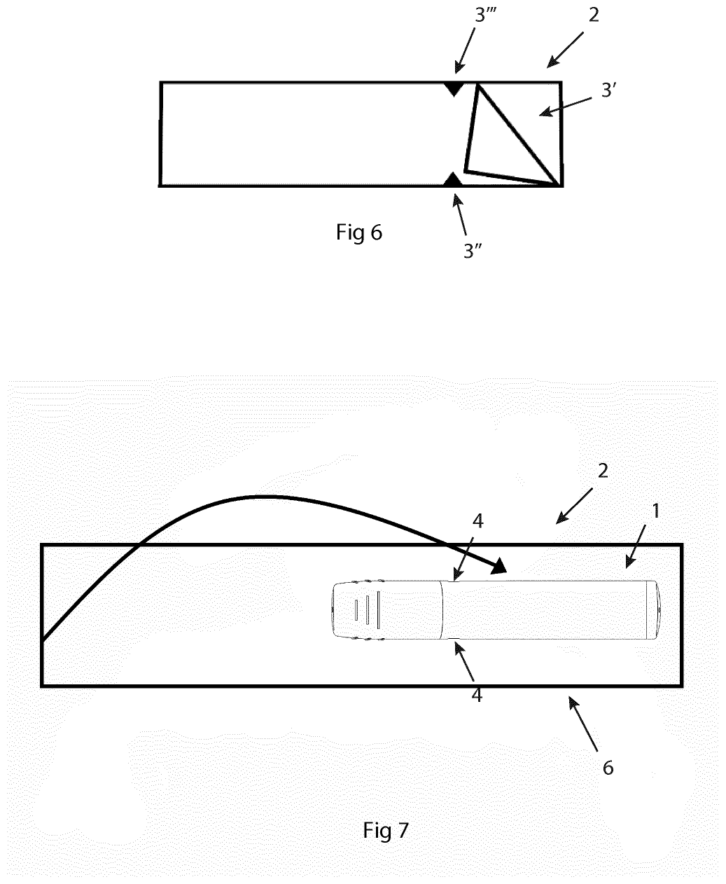

Since safe and fast retrieving of the auto injector may be crucial to a patient, a secondary opening arrangement 3″ may be used as a backup should there be a problem with the primary opening arrangement 3′. Thus, according to some aspects, the opening arrangement 3 comprises a primary opening arrangement 3′ for opening the package 2 and a secondary opening arrangement 3″ for opening the package 2 in case of failure to open with the primary opening arrangement 3′. FIG. 5 shows an example package 2 with a primary and secondary opening. The primary and secondary opening arrangements may be different types of opening arrangements, as shown in FIG. 5, or the same type. For example, the primary opening arrangement 3′ comprises a pre-notch and the secondary opening arrangement 3″ comprises a package with two sides that are torn apart, or in other words, pulled apart, at opening or vice versa as shown in the figure. FIG. 6 also illustrates an example where there are three opening arrangements 3.

There may be an end of the auto injector that is better to grab on to by the user when pulling the auto injector out of the package after opening it. According to some aspects, the package 2 comprises an upper half and a lower half and wherein the auto injector body comprises a first end comprising a cap and a second end, the auto injector being housed in the package 2 such that the first end is located in the lower half of the package 2 and the opening arrangement 3 being arranged on the upper half of the package 2. In other words, the opening arrangement 3 is placed on upper 50% of the pouch. According to some aspects the opening arrangement is placed on the upper 40% of the pouch. According to some aspects the opening arrangement is placed on the upper 30% of the pouch. According to some aspects the opening arrangement is placed on the upper 20% of the pouch. According to some aspects the opening arrangement is placed on the upper 10% of the pouch. This is such that the cap side of the auto injector is not the end that is pulled out first of the package by the user, to prevent that the cap is accidentally opened when extracting the auto injector. When the opening arrangement 3 comprises a primary and a secondary opening arrangement 3 this is more important for the primary arrangement since the secondary is a backup.

According to some aspects, the un-opened package 2 is filled with inert gas and is free of oxygen capturing substance. Not having to use an oxygen capturing substance to capture residual oxygen in the packaging is a consequence of the packaging method herein disclosed. It is preferred that the un-opened package 2 comprises less than 2% oxygen and more preferably less than 1%. Some drugs will have an acceptable shelf life according to this aspect and some drugs may require an oxygen capturing substance such as an oxygen scavenger for an acceptable shelf life. To have a package free of oxygen capturing substance saves cost, gives a simpler packaging process, gives a more robust product and saves material.

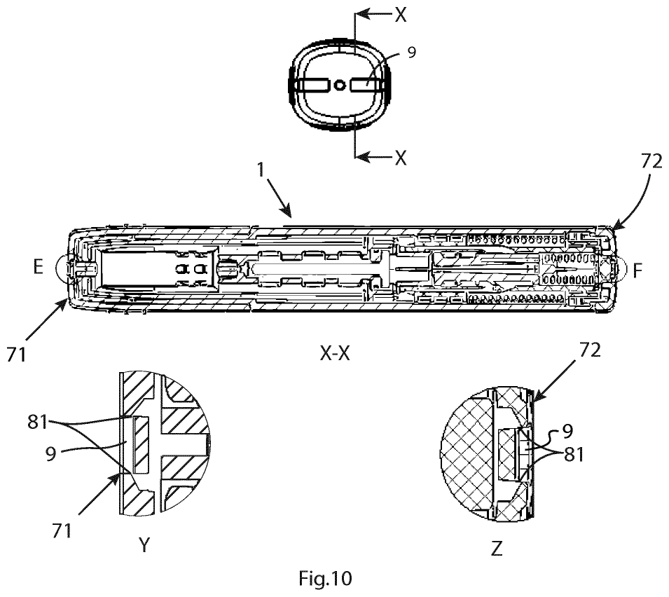

FIG. 10 shows a cut through of an example auto injector where the auto injector has through hole openings 81 in through holes 8 according to below. The through hole openings are in this example arranged in recesses 9 at the end sides. The through hole openings may also be arranged on substantially flat end sides. The top figure shows where in the auto injector the cut through is made, at X. Y and Z are enlarged parts of the middle of the end sides for an enlarged view of the through hole openings 81.

According to some aspects, the auto injector body 7 is elongated having an elongated part, a first end side 71 and a second end side 72 opposite the first end side 71, and at least one through hole 8 is arranged through the auto injector body 8 between the first and second end sides 71, 72, whereby the at least one through hole 8 allow for flow of gas through the auto injector body 7 when the auto injector body 7 houses the medicament container, and the package is a pouch and the inside of the pouch is at least 70 mm longer than the total length of the auto injector and has a maximum width, at the most narrow part of the pouch, of 30 mm wider than the maximum width of the auto injector. An advantage with an auto injector which has through holes between the end sides is that the package can be made much more narrow than conventional packages. This is because air behind the auto injector will be evacuated through the through holes and not via the sides of the auto injector. This reduces the amount of packaging needed and it also makes the packaged auto injector easier to handle for the end user. According to some aspects, the inside of the pouch is at least 80 or 90 mm longer than the total length of the auto injector. This is to avoid wrinkling of the package during the packaging process, which will be further explained below.

The at least one through hole 8 each comprises two through hole openings 81. In other words, each through hole has an opening in each end of the auto injector. It should be noted that if there are more than one through hole, they may share the same opening through the auto injector but have different openings in the end sides.

The package filled with the auto injector may be snugly arranged in a neoprene sleeve such that the movement of the auto injector relative the package is prevented. The neoprene sleeve protects the packaged auto injector from physical damage, keeps the temperature of the auto injector more constant and minimizes movement of the packaged auto injector and thus minimizes friction which can lead to punctures. In the case where the auto injector with through holes and the narrower pouch is used for package, there will not be a lot of packaging material around the auto injector. It is easy to store this packaged auto injector set in a neoprene sleeve; there is not a lot of package that needs to be held in when putting it in the sleeve. The neoprene sleeve protects the packaged auto injector from physical damage, keeps the temperature of the auto injector more constant and minimizes movement of the packaged auto injector and thus minimizes friction which can lead to punctures. The neoprene sleeve is in latex form.

Another word for through hole is gas channel since the through holes are passages for allowing gas to flow through the auto injector body from one end side to the other. In other words, the at least one through hole is an uninterrupted passage for allowing gas to glow through the auto injector from one end side to the other. The through holes, i.e. the gas channels, are holes going through the auto injector body, from one end side to the other, also when the medicament container is in place in the auto injector body. It should be noted that the at least one through hole does not need to be a straight hole through the auto injector, it only needs to be a through hole, or in other words gas channel, between the end sides.

It should be noted that there are numerous different designs of auto injectors which houses different kinds of medicament containers. The disclosure is applicable to all types of auto injectors with prefilled medicament containers. For the narrow pouch, the auto injector is required to have through holes so that air from the pouch can be evacuated through the auto injector.

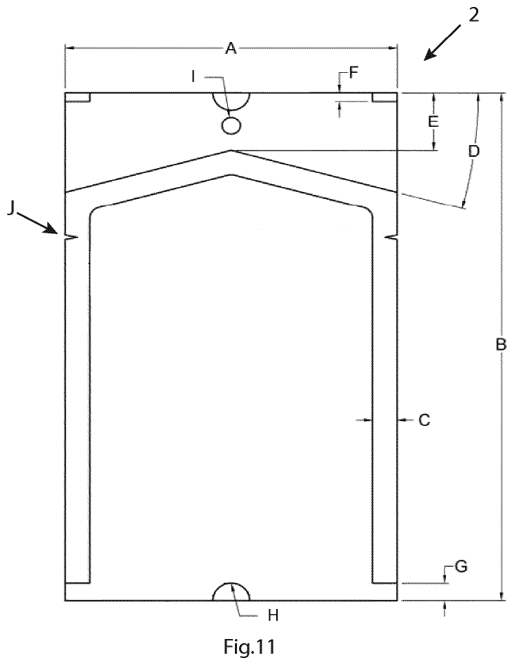

An example package is shown in FIG. 11. The illustration is merely an example and the measurements are to be adapted depending on the size of the auto injector and on which type of method is used for removing air from the package. The example of FIG. 11 is a pouch which has a width A of 95 mm and a length B of 307 mm. This example pouch is for an auto injector with the dimensions 150±1 mm in length and between 23.4 and 27.4±0.5 mm in width. The seal width C is 10 mm and it is a seal between two sides of the pouch. The chevron angle D is 15° and the distance E between the chevron tip and the cut off is 20 mm. The pouch has a tack seal F of 6 mm at the cut off and may have an end gap G at the opposite side. A thumb notch H may be provided for opening the package for retrieving the auto injector. A hang hole I may be provided for hanging the packaged auto injector. The hang hole I is preferably centred and may be 3 mm from the sealed end. In this example, tear notches J are provided on both sides of the package do reduce risk of failure of opening the package to retrieve the auto injector in an emergency. The tear notches J may have a depth of about 1.6 to 2 mm. Peel strength of the illustrated example may be for example around 0.18 kg/cm or 1.0 lb./inch.

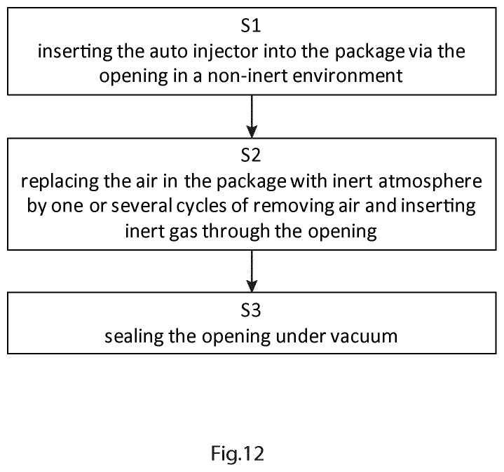

FIG. 12 shows a block diagram of the method. The method comprises a method for filling a package 2 with an auto injector comprising an adrenaline composition, the package 2 comprising an opening. The size of the opening will depend on the type of equipment used for the packaging of the auto injector. The package 2 is for example a square shaped or rectangular pouch and the opening is according to some aspects located on one of the sides.

The adrenaline composition may comprise a chemical oxygen scavenger. Such has been previously discussed. The method comprises inserting the auto injector into the package 2 via the opening in a non-inert environment and, in a non-inert environment and under atmospheric pressure, replacing the air in the package 2 with inert atmosphere by one or several cycles of removing air and inserting inert gas through the opening. The method also comprises sealing the opening under vacuum. This method has a big advantage that it can be performed in a non-inert environment. To keep an inert environment for packaging is very costly and cumbersome. The chemical oxygen scavenger and the process of removing air and filling with inert gas makes it possible to fill the package 2 in a non-inert environment. Another term for removing air and inserting gas is flushing.

The package 2 material is preferably flexible to make the exchange of air against an inert gas, e.g. N2, efficient. A flexible package 2 also allows for pouch integrity control by checking the “vacuumized” state of the pouch. The pouch material should be robust enough to withstand wear and tear of an emergency device during storage and transportation.

According to some aspects, the air is removed using a nozzle type vacuum sealing machine.

An example of such a machine is illustrated in FIG. 15. It has a nozzle 11 that is inserted in the opening of the package 2 and removes the air through the nozzle. This type of machine provides a local point in the opening for removing the air which is an effective way of only removing the air that is in the package 2. A nozzle type vacuum machine is known in the art and has a nozzle for removing air and inserting gas through an opening in the package. The machine has a welding bar 12 that is pressed down on the package to seal it at the end of the packaging process. The welding bar seals the package preferably by heat. The machine also has a seal lip 13, preferably made of rubber, that pushes down on the package on and around the nozzle to keep it sealed when the nozzle is removing air and inserting gas. The seal lip is also pushing down on the package while withdrawing the nozzle to prevent air leakage during the withdrawal. In other words, the seal lip keeps the package closed during removing air and inserting gas and when the process is completed, the welding bar seals the package.

The machine removes air from the pouch while the pouch is subjected to atmospheric pressure. According to some aspects, the pressure of the vacuum should not be under 700 mbar unless the medicament container is bubble free to prevent from plunger movement. When pouching and sealing an auto injector pouch, i.e. package 2, with a nozzle type vacuum welding machine, the pouch opening is made airtight during the gas exchange and sealing process. The access for gas, during pouching, is through the nozzle. This is achieved, for example, by a seal lip, which has been previously described.

The length of the previously discussed package will be affected by the type of machine used to remove the air. In a nozzle type vacuum machine, the distance between the nozzle and the auto injector affects the efficiency of air removal. A distance between 15-30 mm, and preferably between 20-25 mm is efficient when removing air. A shorter distance may lead to problems when sealing the package such that it is not vacuum sealed. A longer distance may lead to inefficient air removal.

When the auto injector, i.e. AI, is inside the pouch and is to be purged with inert gas, e.g. N2, and vacuum-pouched with a nozzle type vacuum sealer/welder, there are two major challenges:

1) When the AI (inside the pouch) is placed too close to the end of the nozzle then, the pouch opening is forced open, by the height of the AI, in an example case 24.4 mm, in proximity of the sealing/welding bar. Thus, no robust sealing process is possible, because the risk for wrinkles, due to the forced widening of the pouch opening, is too great.

2) When the AI inside the pouch is placed too far away from the nozzle ending, then the pouch prevents proper emptying of the pouch through the nozzle, since the pouch is closed between the AI and the nozzle during the sucking cycle. This is negative, since the necessary negative pressure (for pouch integrity control by the user) cannot be achieved and the purging of O2 and replacement with N2 becomes inefficient.

For an AI of the dimensions of 150±1 mm in length and 23.4±0.5 mm in width, it has been discovered that an optimal distance of AI and nozzle lies between 20 and 25 mm. Acceptable results might be achievable also for a wider range, 15-30 mm.

For an AI of greater width, e.g. 40 mm, a greater distance between AI and nozzle can be maintained, e.g. 30-40 mm. This ascertains that the pouch can be sealed/welded with acceptable risk for seam wrinkles; also the pouch is opened wider preventing that the nozzle opening is “sealed” during the sucking cycle.

For an AI of lesser width, e.g. 18 mm, a shorter distance between AI and nozzle can be maintained, e.g. 15-20 mm. This ascertains that the pouch can be sealed/welded with acceptable risk for seam wrinkles; there is enough distance between sealing bar and pouch-spreading device; also the pouch is opened less, therefore a shorter distance is sufficient to prevent that the nozzle opening is “sealed” during the sucking cycle.

Further, the following parameters are relevant:

The distance between the AI and the outer edge of the sealing/welding bar: This may be at least 5-6 cm distance from the AI inside the pouch. This parameter is connected to the seal-wrinkle issue. If the sealing bar is too close to the AI of a width of 24.4 mm, then seal wrinkles, and thus leakage, is a major risk and no robust sealing process is possible. Longer distances between the sealing/welding bar and the AI can be acceptable, but then the nozzle length should be adjusted to control the distance between the nozzle opening and the AI.

The vacuum purging and sealing process works more reliably if the seal lip, where a seal lip is of rubber like material and pushes the two sides of the pouch together, during suck/blow cycles and also when the nozzle is pulled out of the pouch right before welding, is positioned on the opposite side, relative to the AI, of the sealing bar to avoid sealing wrinkles. Also, the pouch may leak during the vacuum cycles if the lip is on the AI side and thus too close to the widening effect of the AI. Consequently the pouch is, according to some aspects, long enough to fit the AI, to allow enough distance between AI and nozzle opening as previously described, to allow enough distance between Al and sealing bar and to allow that the open, unsealed side of the pouch, on the “other” side of the sealing/welding bar relative to the AI, stretches far enough to be completely covered by the sealing seal lip.

Exact dimensions of the pouch depend on the dimensions of the sealing/welding bar and the seal lip.

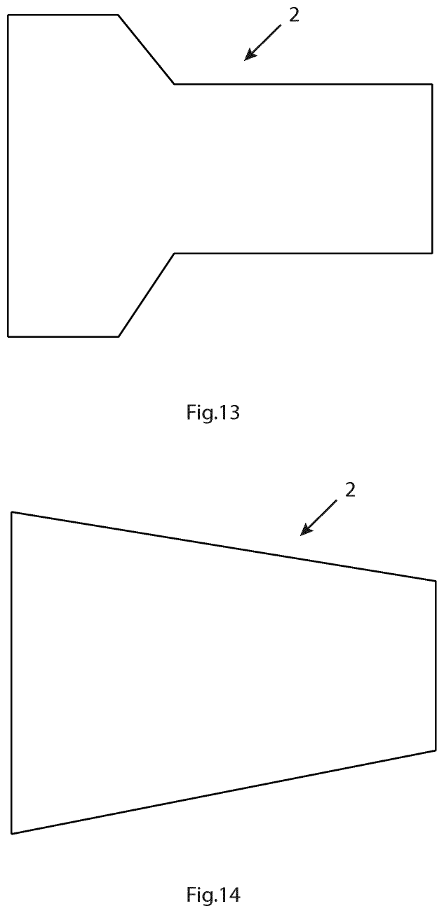

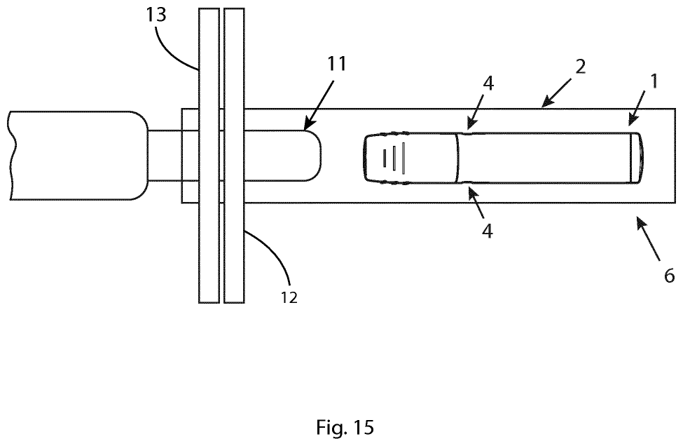

Pouch dimensions: The width of the open side of the pouch, that is being sealed/welded at the end of the vacuum pouching process, may have a minimum width of 80-100 mm. The pouch can have this width over its complete length. However, if pouch material is to be minimized then a pouch can be used that has an unsealed opening of 80-100 mm and that narrows to the AIs width, for example ca 30-40 mm, in sufficient distance to the sealing/welding bar. Two examples of how the pouch may be shaped are shown in FIGS. 13 and 14. In FIG. 13, the opening is two the left and there is a tapering section for reducing the width of the pouch. In FIG. 14, the whole pouch is tapered, from the opening to the left to the other end.

An example of the packaging process will now be described with example devices.

Step 1: Pouching; consisting of inserting the auto injector into the pouch, which is sealed on all sides but one

Step 2: Replacing air in the pouch with inert atmosphere, e.g. nitrogen, in one or several cycles of sucking air and blowing e.g. nitrogen

Step 3: Vacuum sealing.

The pouching achieves:
Removal of “enough” oxygen from the inside of the pouch and the auto injector and replacing it with inert gas. The more oxygen is removed during this process, the longer shelf life can be achieved. Data shows that 36 M of shelf life can be achieved when reaching about <=1 or 2% oxygen in the pouch. This also allows for storage of an auto injector comprising adrenaline for longer than one year at 40° C.
Sealing of the pouch in a vaccumized look, it should be clearly visible that hardly any gas is in the pouch except for the inert gas that is inside the body of the auto injector; i.e. no bubbles in the package 2. It also allows the patient to check pouch integrity. If the pouch loses the vaccumized look, a hole can be assumed and the product has to be replaced. This procedure allows easy packaging integrity control without e.g. O2 sensor inside the pouch (which would be an alternative approach but which makes packaging much harder since packaging under inert gas should not trigger O2 chemical sensor).
If having a negative pressure in the pouch/AI, gas from the gas bubble inside the primary packaging will actively (driven by pressure difference in the primary packaging (e.g. 1 bar) and the pouch (e.g. 700 mbar)) permeate to the outside of the primary packaging and thus A) reduce the amount of residual oxygen B) reduce the size of the air bubble inside and thus risk for accidental movement during air travel.
For primary containers that contain a gas bubble: avoiding of an excess of negative pressure:
   during pouching—to avoid, or minimize, plunger movement which may affect sterility of the drug.

Vacuum chamber pouching is unsuitable to achieve the above listed requirements. It requires too low negative pressure during pouching for an acceptable vacuumed look of the pouch, which might lead to plunger movement. A here desired pouching process removes air from the pouch while the pouch is subjected to atmospheric pressure; which is disclosed in the method discussed above.

An example of the process of providing a packaged auto injector will now be described in more detail.

1) Pre-fillable syringes are filled with drug product. Syringe is not a gas tight system (gas tight plungers are not available and the needle shield is by design permeable for gas- to allow sterilization by ethyleneoxide

2) Filled syringes are stored 10-12 weeks

3) After release, ca 12 weeks after filling, syringes will be assembled into auto injectors, the auto injectors are labelled and vacuum pouched:
a. Pouches, i.e. packages, have been optimized in dimensions. Pouches may have transparent part to allow visual control of drug
   i. Pouches are only slightly wider than the Auto injectors to minimize bulkiness of pouched auto injector. At the side of the pouch where the nozzle is to be inserted, the pouch is wider, it may for example be wider than 80 mm.
   ii. Pouches being so narrow requires ventilation through the auto injector (1 to change atmosphere inside auto injector and 2 to allow gas flow from the opposite end of the narrow pouch)
   iii. Pouches is significantly longer than device (ca 70 mm or more) because narrow pouches are forced open by auto injector body; this may make the welding of the open side of the pouch unreliable (wrinkles lead to leaks of closing weld seam)
b. Pouches are atmosphere exchanged by nozzle type vacuum welding machine. Only nozzle type allows the two following requirements
   i. Vacuum during and after pouching process is never <500 mbar (otherwise plunger movement could lead to loss of sterility)
   ii. Vacuum after pouching is between 500 mbar and ca 750 mbar (TBC) to allow vacuumed feel and look of pouch (regulatory requirement to be able to control pouch integrity for end user)
c. After completed pouching, the pouched auto injectors are packed in carton together with neoprene sleeve as accessory

Neoprene sleeve is to protect pouch during transport and throughout shelf life (3 years). The neoprene sleeve sits tight around pouched auto injector which minimizes movement and thus friction and thus risk for puncture. It also dampens fall and temperature changes during end user transport.

### REFERENCE LIST

1. Auto injector

2. Package

3. Opening arrangement

4. At least two apertures

5. Blocking arrangement

6. Packaged auto injector set

7. Auto injector body
71. First end side
72. Second end side

8. At least one through hole
81. At least one through hole opening

9. Recess

10. Grip

11. Nozzle

12. Welding bar

13. Seal lip

1. A packaged auto injector set for providing a packaged auto injector with adrenaline comprising:
an at least partly transparent medicament container comprising an adrenaline composition,
a hollow auto injector body defining a space which houses the medicament container and thereby forming the auto injector, and
a non-oxygen permeable package for housing the auto injector and including an opening arrangement for opening the package to retrieve the auto injector,
wherein the auto injector body is provided with at least two apertures disposed on opposite sides of the medicament container allowing for visual control of the adrenaline composition inside the container, at least a part of the package is transparent to visible light to allow visual control of the adrenaline composition through the apertures, and the packaged auto injector set comprises at least one blocking arrangement, which is non-transparent to at least ultraviolet light, arranged such that ultraviolet light is prevented from entering said at least two apertures.

2. The packaged auto injector set according to claim 1, wherein the at least one blocking arrangement is arranged on the auto injector body and comprises at least one film placed over the at least two apertures, wherein the film is transparent to visible light and non-transparent to ultraviolet light.

3. The packaged auto injector set according to claim 1, wherein the at least one blocking arrangement is arranged on the part of the package which is transparent to visible light and comprises a layer in the transparent part which is transparent to visible light and non-transparent to ultraviolet light.

4. The packaged auto injector set according to claim 1, wherein the at least one blocking arrangement is arranged on the package and wherein the transparent part of the package is closable such that the package is non-transparent to visible light and ultraviolet light when the transparent part is closed and thus blocks visible light and ultraviolet light from entering the at least two apertures.

5. The packaged auto injector set according to claim 4, wherein the transparent part is arranged on a first side of the package and the package has a second side, which is non-transparent to ultraviolet light and visible light, wherein the blocking arrangement comprises a folding of the second side of the package over the first side of the package to cover the transparent part and thus blocking ultraviolet light and visible light from entering the at least two apertures.

6. The packaged auto injector set according to claim 5 wherein the opening arrangement is located such that it is not covered when the package is folded.

7. The packaged auto injector set according to claim 4, wherein the at least one blocking arrangement comprises a flap arranged over the transparent part, whereby the flap is adapted to be lifted to allow visual control of the medical composition.

8. The packaged auto injector set according to claim 1, wherein the opening arrangement is arranged to be torn by a user such that the package tears, and wherein the opening arrangement comprises:
a stop that prevents the user from tearing the package all the way through, such that the package is kept in one part when teared.

9. The packaged auto injector set according to claim 1, wherein the opening arrangement comprises a primary opening arrangement for opening the package and a secondary opening arrangement for opening the package in case of failure to open with the primary opening arrangement.

10. The packaged auto injector set according to claim 1, wherein the un-opened package is filled with inert gas and is free of oxygen capturing substance.

11. The packaged auto injector set according to claim 1, wherein the adrenaline composition comprises a chemical oxygen scavenger.

12. The packaged auto injector set according to claim 1, wherein the auto injector body is elongated having an elongated part, a first end side and a second end side opposite the first end side, and at least one through hole is arranged through the auto injector body- between the first and second end sides, whereby the at least one through hole allow for flow of gas through the auto injector body when the auto injector body houses the medicament container, and the package is a pouch and the inside of the pouch is at least 70 mm longer than the total length of the auto injector and has a maximum width, at the most narrow part of the pouch, of 30 mm wider than the maximum width of the auto injector.

13. The packaged auto injector set according to claim 1, wherein the package filled with the auto injector is snugly arranged in a neoprene sleeve such that the movement of the auto injector relative the package is prevented.

14. A method for filling a package with an auto injector according to claim 1 comprising an adrenaline composition, the package comprising an opening, wherein the method comprises:
inserting the auto injector into the package via the opening in a non-inert environment, and
in a non-inert environment and under atmospheric pressure:
replacing the air in the package with inert atmosphere by one or several cycles of removing air and inserting inert gas through the opening,
and
sealing the opening under vacuum.

15. The method according to claim 14, wherein the air is removed using a nozzle type vacuum sealing machine.
